(19) United States
(12) Patent Application Publication
(10) Pub. No.: US 2004/0228931 A1
(43) Pub. Date: Nov. 18, 2004

(54) Novel composition for hormonal balance and uses thereof

(75) Inventors: Dilip Chokshi, Parsippany, NJ (US); Mitch Skop, Closter, NJ (US)

Correspondence Address: HOFFMANN & BARON, LLP, 6900 JERICHO TURNPIKE, SYOSSET, NY, 11791, US

(73) Assignee: Pharmachem Laboratories, Inc.

(21) Appl. No.: 10/435,949

(22) Filed: May 12, 2003

(51) Int. Cl. A61K 35/78; A61K 9/68; A61K 9/20

(52) U.S. Cl. 424/757; 424/765; 424/764; 424/48; 424/440

(57) ABSTRACT

The present invention provides a composition and methods of using composition consisting essentially of a soy component, chasteberry, clover, and black cohosh, in a suitable carrier, optionally with each of the soy component, chasteberry, clover, and black cohosh is enrobed in a glycoprotein matrix.

### BACKGROUND OF THE INVENTION

The present invention relates to the mitigation of symptoms associated with female reproductive hormones.

During a woman's reproductive years, the ovaries produce hormones such as estrogen and progesterone. Hormones are necessary to induce ovulation and prepare the body for a successful pregnancy. However, with age, a woman's body gradually stops releasing hormones from the ovaries. This period of time is referred to as menopause.

During menopause, the woman can experience wide fluctuations in baseline hormonal levels. The erratic hormone levels associated with menopause can cause a wide variety of symptoms. These symptoms can last from six months to two years. The symptoms can include hot flashes, sweating, heart palpitations, headaches, cramping, nausea, and mood swings.

Eventually, the woman's body essentially stops releasing hormones. As a result, the woman becomes susceptible to certain conditions. For example, the body's ability to maintain calcium levels is compromised which results in an increased loss of minerals from the bones (e.g., osteoporosis).

In addition to menopause, there are numerous other conditions that can have an effect on a woman's hormonal balance. For example, thyroid disease, stress, endometriosis, and cancer can effect a woman's hormonal balance is such a way that menopausal-type symptoms become present.

Many of the symptoms of hormone imbalance and/or decrease in hormones can be relieved with hormone replacement therapy (HRT). The use of HRT, such as synthetic estrogens and/or progesterones, can be effective in suppressing the symptoms of menopause, such as hot flashes, and may be effective in reducing calcium loss, and preserving bone density.

However, side effects such as, for example, mastalgia, edema, abdominal bloating, and increase in the size of uterine leiomyomata have been associated with the use of HRT. Importantly, cancers, such as breast cancer and endometrial cancer have also been associated with HRT.

Many women that use HRT, and their clinicians, struggle with determining suitable dosages of the synthetic hormones. The difficulties with finding an effective dose that does not present the unwanted side effects contributes greatly to women ceasing HRT entirely.

In addition, due to the side effects associated with HRT, many women are hesitant about using “synthetic” pharmaceutical drugs such as HRT drugs. Therefore, many women in need of regulating hormonal balance today are searching for “natural alternatives” to hormone replacement therapy (HRT) to alleviate their symptoms and protect against other health conditions, such as osteoporosis.

Several prior art references disclose herbal compositions for hormonal balance. For example U.S. Pat. No. 6,242,012 to Newmark et al. disclose a herbal composition containing dong quai root extract, schizandra berry extract, organic ginger rhizome, black cohosh root and rhizome extract, chaste tree berry extract, and rosemary leaf and essential oil extract.

However, it has been reported that dong quai is not recommended for certain individuals such as, for example, diabetics since it can increase blood sugar levels.

U.S. Pat. No. 6,497,885 to Trant discloses a composition containing Vitex agnus-castus (chasteberry), antioxidants, L-arginine, folic acid, vitamin B6, iron, zinc, and magnesium.

However, many women avoid supplements that contain iron because of the constipating effects associated with iron-containing products.

There is a need for a herbal composition that can effectively regulate hormonal balance in a woman in need thereof without containing ingredients that have adverse side effects and/or risks.

Moreover, the individual ingredients in the above prior art references can lose their bioactivity and stability during manufacturing, for example, due to high heat and/or compression into tablet form. Thus, the bioavailability of the ingredients is rendered insufficient for promoting hormonal balance. As a result, a larger amount of the ingredients must be ingested to gain the beneficial effects.

Thus, there is a need for natural compositions that are stable, concentrated, and bioavailable that effectively promote hormonal balance and relieve symptoms associated with hormonal imbalance in women.

The present invention addresses the above need by providing a composition containing specifically selected ingredients that have minimal adverse side effects and/or risks, and that can be optionally enrobed in a glycoprotein matrix, for promoting hormonal balance.

### SUMMARY OF THE INVENTION

These and other objective have been met by the present invention by providing a composition consisting essentially of a soy component, chasteberry, clover, and black cohosh, in a suitable carrier.

In another embodiment, the invention provides a composition consisting essentially of a soy component, chasteberry, clover, and black cohosh, in a suitable carrier, wherein each of the soy component, chasteberry, clover, and black cohosh is enrobed in a glycoprotein matrix.

In yet another embodiment, the invention provides a method for promoting hormonal balance in a woman in need thereof. The method comprises administering to the woman an effective amount of a composition consisting essentially of a soy component, chasteberry, clover, and black cohosh, in a suitable carrier.

In a further embodiment, the invention provides a method for promoting hormonal balance in a woman in need thereof. The method comprises administering to the woman an effective amount of a composition consisting essentially of a soy component, chasteberry, clover, and black cohosh, in a suitable carrier, wherein each of the soy component, chasteberry, clover, and black cohosh is enrobed in a glycoprotein matrix, and

### BRIEF DESCRIPTION OF THE INVENTION

In accordance with the present invention, a composition is provided which consists essentially of a soy component, chasteberry, clover, and black cohosh in a suitable carrier. The composition promotes hormonal balance in a woman in need thereof.

The soy component can be any component derived from a soybean. Preferably, the soybean is non-genetically modified (non-GMO) organic soybean. “Organic” soy as used herein means that the soy component is derived from soybeans that are grown without synthetic pesticides and fertilizers.

Soybeans typically contain high levels of isoflavones and protein. An example of a soy component useful in the present invention are soy proteins. Preferably, the soy component is isoflavone. Isoflavones belong to the family of phytoestrogens, which are compounds that typically mimic estrogen.

A suitable soy component is also defatted, non-genetically engineered (non-GMO), organic soy flour.

Chasteberry is a berry obtained from a chaste tree. Chasteberry is botanically known as Vitex agnus-castus. Other common names for chasteberry include, for example, vitex, monk's pepper, and chaste tree berry. Chasteberry is a medicinal herb which generally contains phytohormones. The herb typically influences hormonal activity by stimulating the pituitary gland to produce more luteinizing hormone.

Preferably, the chasteberry component contains a combination of one or more of iridoids, flavanoids and terpenoids.

Clover is a plant that commonly grows in Europe and North America. The clover can be any variety of clover, such as red clover and white clover. Preferably, the clover is red clover. Red clover is botanically known as Trifolium pretense and contains high levels of isoflavone compounds, such as genistein.

Black cohosh is a perennial plant that is a member of the buttercup family. Black cohosh is botanically known as Actaea racemosa and Cimicifuga racemosa. Other common names for black cohosh include black snakeroot, bugbane, bugwort, rattleroot, rattletop, rattleweed, and macrotys. Preparations of black cohosh are typically made from roots and rhizomes.

Preferably, the black cohosh component contains terpenoids and/or flavanoids.

The active component of soybean, chasteberry, red clover, and black cohosh can be from any part of the plant (e.g., stem, leaves, flowers, seed, roots, or rhizome). It is known in the art where the active component is located in soybean, chasteberry, red clover, and black cohosh. For example, the active component is preferably obtained from the roots and rhizome of black cohosh, and from the flowers of red clover.

The active component can be extracted and concentrated into a liquid or solid form. The extraction and concentration of the active component of soybean, chasteberry, red clover, and black cohosh can be done by any method known to those skilled in the art. For example, the active component can be extracted using supercritical extraction methods. Such methods are disclosed in, for example, U.S. Pat. Nos. 5,932,101 and 5,120,558 and are hereby incorporated by reference. Briefly, supercritical extraction involves the use of a supercritical fluid, such as carbon dioxide, for extraction.

Although preferred, it is not crucial to extract and concentrate the active component from soybean, chasteberry, red clover, and black cohosh. For example, parts of the plant (e.g., soybean, chasteberry, red clover, or black cohosh) containing the active component can be dried and used in the composition of the present invention. For instance, the roots and rhizome of black cohosh can be dried and ground into a powder for use in the composition.

The composition can contain essentially any amount of soybean, chasteberry, red clover, and black cohosh as desired. For example, the percentage of soybean, chasteberry, red clover, and black cohosh can vary between 0.1 and 99.9% by weight of the composition.

“Consisting essentially of” as used herein means that the composition contains no other ingredients that can promote hormonal balance. Thus, other components such as stabilizers, surfactants, salts, buffering agents, etc., described below, can be included in a formulation (e.g., tablets, gelatin capsules, pills, troches, elixirs, caplets, powders, granules, sachets, suspensions, syrups, wafers, chewing gum) of the composition.

The composition can be formulated with suitable carriers, such as inert, inorganic or organic carriers. Suitable carriers include, for example, lactose, corn starch or derivatives thereof, talc, stearic acid or its salts, vegetable oils, waxes, fats, semi-solid and liquid polyols and the like, water, polyols, saccharose, invert sugar, glucose and the like, alcohols, and glycerin.

In another embodiment, a composition is provided which consists essentially of a soy component, chasteberry, clover, and black cohosh in a suitable carrier, wherein each of the ingredients (i.e., soy component, chasteberry, clover, and black cohosh) is enrobed in a glycoprotein matrix.

The glycoprotein matrix and ingredient can be associated with each other physically and/or chemically, such as by chemical reaction, and/or secondary chemical bonding, e.g., Van der Waals forces, hydrogen bonds, etc. It is believed that glycoprotein matrix associates with the ingredient by weak covalent bonds. Due to the association between the glycoprotein matrix and the ingredient, the ingredient is enrobed (e.g., coated, encapsulated) with a glycoprotein matrix.

The glycoprotein matrix is the glycoprotein to which the ingredient is enrobed. Glycoprotein is a composite material typically made of one or more carbohydrate groups and a simple protein. A glycoprotein matrix is a molecular network comprised of a plurality of glycoprotein molecules bound together.

The carbohydrate in the glycoprotein can be any suitable carbohydrate, such as a monosaccharide, disaccharide, oligosaccharide, or polysaccharide. Oligosaccharide is preferred. The protein of the glycoprotein can any suitable polypeptide. The ratio of carbohydrate to protein in the glycoprotein matrix can vary, for example, from 99:1 to 1:99 by weight. A ratio of approximately 1:1 is preferred.

The ratio of glycoprotein matrix to the ingredient can also vary. It is preferred that the ratio of glycoprotein matrix to the ingredient will be such that all or nearly all of the ingredient is bound by glycoprotein matrix. Such amounts can be readily determined by the skilled practitioner.

To ensure that essentially all of the ingredient is bound, higher ratios of glycoprotein matrix to ingredient can be used. The invention also contemplates that there may be insufficient glycoprotein to bind all of the ingredient. In such cases, the ratio of glycoprotein matrix to ingredient can be less.

The glycoprotein matrix is derived from one or more strains of microorganisms A microorganism is typically any microscopic organism, such as bacteria, algae, fungi, and protozoa. The microorganisms can be naturally occurring strains of laboratory-bred strains. Microorganisms that produce a glycoprotein matrix include, but are not limited to, yeast (e.g., fungi) and bacteria. A preferred yeast is Saccharomyces cervisiae.

Bacteria useful in the present invention for producing glycoprotein matrix can be any bacteria. Preferably, the bacteria functions as a probiotic. A probiotic is typically a microbial supplement which beneficially improves intestinal microbial balance. For example, probiotics can aid in digestion and can help prevent illness by promoting the growth of good bacteria in a digestive host.

Bacteria which are useful as probiotics include probiotics within the genus Lactobacillus. For example, such probiotics include, but are not limited to, Lactobacillus acidophilus, Lactobacillus bulgaricus, Lactobacillus caucasicus, and Bacterium bifidus. Preferred probiotics include Lactobacillus acidophillus and Bacterium bifidus.

One or more strains of microorganisms can be used provided that at least one of the microorganisms produces glycoprotein. When using combinations of microorganisms, the growth of one type of microorganism should not prevent the growth of the other. For example, various types of different yeast that produce glycoprotein can be used. Also, yeast and bacteria can be combined to produce glycoprotein. This combination is particularly advantageous because various types of bacteria, such as Lactobacillus acidophillus, also produce glycoprotein.

As discussed more specifically below, an ingredient can be enrobed with a glycoprotein matrix by allowing the microorganism to ferment, in the presence of the ingredient. As used herein, fermentation is the process by which microorganisms metabolize raw materials, such as amino acids and carbohydrate, to produce glycoprotein.

The microorganisms produce glycoprotein both intracellularly and extracellularly The intracellular glycoprotein will mainly be located in the cytoplasm of the microorganism or become part of the microorganism's physical structure. The glycoprotein from the microorganism that forms the glycoprotein matrix is mainly extracellular and, therefore, is available to be bound to the ingredient. Intracellular glycoprotein can also be made accessible for binding to ingredient by rupture of the microorganisms after glycoprotein production. At the end of the manufacturing process of enrobing an ingredient with a glycoprotein matrix, these microorganisms are usually inactive.

Since the ingredients are to be used in a composition that is to be ingested, the microorganism used to produce the glycoprotein matrix should be suitable for consumption by humans. Examples of such microorganisms include Lactobacillus acidophillus and Saccharomyces cervisiae.

The ingredient can be enrobed in a glycoprotein matrix by any method known in the art. Methods for enrobing substances, such as vitamins and minerals, with a glycoprotein matrix are disclosed in U.S. patent application Ser. Nos. 09/906,576 and 09/757,222. The specification of U.S. patent application Ser. Nos. 09/906,576 and 09/757,222 are hereby incorporated by reference.

For example, a method for enrobing an ingredient with a glycoprotein matrix includes contacting the ingredient with a glycoprotein producing microorganism under conditions in which the microorganism produces glycoprotein.

The microorganisms require a medium in which to ferment and produce glycoprotein. Such media are known to those skilled in the art, and are usually liquid. Water is preferred. The microorganism solution should contain enough growth medium so as to allow for efficient growth of the microorganisms, as is known in the art. When the microorganisms are added to the liquid medium, a microorganism solution is formed.

A microorganism solution is prepared in which the microorganisms will produce glycoprotein. The number of colony forming units of microorganism added to the medium will vary based upon the type of microorganism used.

A sufficient amount of colony forming units should be added to the microorganism solution to enrobe at least some of the ingredient. If the composition of the invention is to contain a small amount of ingredient, fewer microorganisms will be required to bind the ingredient with glycoprotein matrix. It is preferred that enough colony forming units be added to the microorganism solution to bind essentially all of the ingredient with glycoprotein matrix. One skilled in the art can determine such amounts.

The amount of colony forming units of the microorganism utilized can also depend upon the molecular weight and amount of the ingredient in the composition. For example, more colony forming units are necessary to fully bind a higher molecular weight ingredient. Fewer colony forming units are required for a low molecular weight ingredient.

The microorganisms that produce the glycoprotein typically require nutrients to efficiently grow, multiply, and form glycoprotein by metabolizing the nutrients. The nutrients can be directly added to the microorganism solution or can be added to a nutrient media, which is then added to the microorganism solution.

Amino acids are one nutrient that may be necessary for efficient glycoprotein production. The amino acids are metabolized by the microorganisms and ultimately become part of the polypeptide within the glycoprotein matrix. The amino acids should include those that are suitable for the manufacture of glycoprotein. Such amino acids include, but are not limited to, glutamine, lysine, cysteine and methionine, aspartic acid, leucine, valine, alanine, arginine, and glycine. The amino acids need not be in a pure form, but can be added as part of a stable compound. Examples of amino acid compounds that can be used are L-Glutamic Acid, L-Lysine HCl, L-Cysteine HCl and DL-Methionine.

The amount of amino acids will vary based upon the amount, molecular weight, and percentage of ingredient desired to be enrobed by glycoprotein matrix. If the ingredient is a small molecular weight molecule, it may not be necessary to add amino acids as a nutrient for the production of glycoprotein matrix by the microorganisms.

Carbohydrate is a nutrient that is added for the efficient production of glycoprotein by the microorganism. As with the amino acids, the carbohydrate can be added to a nutrient media, which is then added to the microorganism solution, or can be added directly to the microorganism solution. Carbohydrates beneficial for the production of glycoprotein are known in the art. The carbohydrate can be, for example, a polysaccharide, oligosaccharide, disaccharide or monosaccharide or combinations thereof Examples of appropriate carbohydrates include, but are not limited to, maltose and gum acacia. Maltose is most preferred.

The amount of carbohydrate added to the nutrient media or microorganism solution will vary depending upon the complexity and molecular weight of the carbohydrate added to the solution. The amount of carbohydrate should be sufficient to permit the microorganisms to produce the glycoprotein matrix. The amount of carbohydrate necessary will also vary based upon the amount and percentage of ingredient desired to be bound by glycoprotein matrix.

Enrobing an ingredient occurs in the microorganism solution as the glycoprotein is being produced by the microorganisms. Thus, the microorganism solution will contain the ingredient to be bound by glycoprotein matrix. The ingredient is added before or after fermentation of the microorganisms begins. If the ingredient is added after fermentation, it is preferred that the ingredient is added immediately after fermentation of the microorganisms begins.

Applicants believe that the ingredients enrobed in a glycoprotein matrix will exhibit superior bioactivity and stability as compared to conventional forms of the ingredients. The improved bioactivity and stability will provide the woman with greater benefits from the ingredients than that obtained from conventional forms.

In another embodiment, the invention provides a method for promoting hormonal balance in a woman in need thereof, by administering to the woman an effective amount of a composition described above.

The term “effective amount” as used herein is the amount that is sufficient for promoting hormonal balance (e.g., alleviate symptoms, described above, of hormonal imbalance). The effective amount can be determined during pre-clinical trials and clinical trials by methods familiar to physicians and clinicians.

An effective amount, preferably in a pharmaceutical composition, may be administered to a woman in need thereof by any of a number of well-known methods for administering pharmaceutical compounds.

The soybean component, chasteberry, clover, and black cohosh can be mixed with a suitable pharmaceutical carrier (vehicle) or excipient as understood by practitioners in the art. Examples of carriers and excipients include starch, milk, sugar, certain types of clay, gelatin, lactic acid, stearic acid or salts thereof, including magnesium or calcium stearate, talc, vegetable fats or oils, gums and glycols.

The composition may be administered to a woman by sustained release, as is known in the art. Sustained release administration is a method of drug delivery to achieve a certain level of the drug over a particular period of time. The level typically is measured by serum concentration.

Any formulation known in the art of pharmacy is suitable for administration of the composition. Some examples of formulations include tablets, gelatin capsules, pills, troches, elixirs, caplets, powders, granules, sachets, suspensions, syrups, wafers, chewing gum, soft chews, and the like. For oral administration, liquid or solid formulations, may be used.

In a preferred embodiment, the ingredients are in a soft chew. Soft chews are soft, chewable confections that have a nougat candy consistency. For example, the confection imparts a soft, yet unsticky chew texture. Such a confection can be obtained by any known method, so long as the use of high heat, excessive moisture and dehydration processes are avoided.

For example, a suitable method for producing such a soft chew confection is disclosed in U.S. Pat. No. 6,517,886, assigned to Biovail Corp Int'l, which is herein incorporated by reference in its entirety. The confection can be dispensed as individually wrapped pieces or in a scored bar that can be broken off and consumed as an individual piece.

Applicants believe that a composition of the invention in a soft chew will be more potent. Unlike tablet formulations, by avoiding the use of high heat, excessive moisture and compression, the ingredients in the soft chew are minimally disturbed. Therefore, the soft chew will provide the ingredients in a more bioactive form.

For systemic, topical, intranasal, or subcutaneous administration, formulations of the composition may utilize conventional diluents, carriers, or excipients etc., such as are known in the art can be employed. For example, the formulations may comprise one or more of the following: a stabilizer, a surfactant, preferably a nonionic surfactant, and optionally a salt and/or a buffering agent. The composition may be delivered in the form of an aqueous solution, or in a lyophilized form.

The stabilizer may, for example, be an amino acid, such as for instance, glycine; or an oligosaccharide, such as for example, sucrose, tetralose, lactose or a dextran. Alternatively, the stabilizer may be a sugar alcohol, such as for instance, mannitol; or a combination thereof. Preferably the stabilizer or combination of stabilizers constitutes from about 0.1% to about 10% weight for weight of the composition.

The surfactant is preferably a nonionic surfactant, such as a polysorbate. Some examples of suitable surfactants include Tween20, Tween80; a polyethylene glycol or a polyoxyethylene polyoxypropylene glycol, such as Pluronic F-68 at from about 0.001% (w/v) to about 10% (w/v).

The salt or buffering agent may be any salt or buffering agent, such as for example, sodium chloride, or sodium/potassium phosphate, respectively. Preferably, the buffering agent maintains the pH of the pharmaceutical composition in the range of about 5.5 to about 7.5. The salt and/or buffering agent is also useful to maintain the osmolality at a level suitable for administration to a human or an animal. Preferably the salt or buffering agent is present at a roughly isotonic concentration of about 150 mM to about 300 mM.

The formulations of the composition may additionally contain one or more conventional additives. Some examples of such additives include a solubilizer such as, for example, glycerol; an antioxidant such as for example, benzalkonium chloride (a mixture of quaternary ammonium compounds, known as “quats”), benzyl alcohol, chloretone or chlorobutanol; anaesthetic agent such as for example a morphine derivative; or an isotonic agent etc., such as described above. As a further precaution against oxidation or other spoilage, the pharmaceutical compositions may be stored under nitrogen gas in vials sealed with impermeable stoppers.

Women in need of promoting hormonal balance include those women who are suffering from hormonal imbalance. Examples of women suffering from hormonal imbalance include, for example, peri-menopausal women, menopausal women, women with erratic menstrual cycles, women suffering from post-partum depression, women in which menstruation has ceased for any reason, including for example hysterectomy, ovarian failure, ovarian removal, chemotherapy, radiation, etc.

### EXAMPLES

### Example 1

### Preparation of Soy+Glycoprotein Matrix (GPM) Complex

This example demonstrates the preparation of soy plus glycoprotein matrix (GPM) complex to yield a soy+GPM complex. The method employs preparing, in a first container, an aqueous solution of soy and adding a peptone made of amino acids.

In a second container an active yeast solution is prepared. Active baker's yeast, Saccharomyces cervisiae is added to water to form an aqueous solution. Maltose and gum acacia are then added.

The first container containing the soy is then inoculated very slowly into the active yeast solution to form a live fermented solution. The mixture is allowed to ferment for four to six hours. To promote yeast growth, plant proteins and carbohydrates are added. Proteolytic enzyme, such as papain, is then added.

Lactobacillus acidophillus is added to the live fermented solution and allowed to ferment for about 2 hours. Active fermentation is then stopped by heating the solution to 160-170° F. for three hours.

The fermented solution containing soy is then homogenized in a shearing pump (Charles Ross & Sons Corp.) for approximately 1-2 hours and spray dried (NIRO, Nicholas Engineers Research Corp.) for approximately 4 hours. The resulting product is a powder containing the soy+GPM complex.

### Example 2

### Preparation of Chasteberry+Glycoprotein Matrix (GPM) Complex

This example demonstrates the preparation of chasteberry plus glycoprotein matrix (GPM) complex to yield chasteberry+GPM complex. The method employs preparing, in a first container, an aqueous solution of chasteberry and adding a peptone made of amino acids.

In a second container an active yeast solution is prepared. Active baker's yeast, Saccharomyces cervisiae is added to water to form an aqueous solution. Maltose and gum acacia are then added.

The first container containing the chasteberry is then inoculated very slowly into the active yeast solution to form a live fermented solution. The mixture is allowed to ferment for four to six hours. To promote yeast growth, plant proteins and carbohydrates are added. Proteolytic enzyme, such as papain, is then added.

Lactobacillus acidophillus is added to the live fermented solution and allowed to ferment for about 2 hours. Active fermentation is then stopped by heating the solution to 160-170° F. for three hours.

The fermented solution containing chasteberry is then homogenized in a shearing pump (Charles Ross & Sons Corp.) for approximately 1-2 hours and spray dried (NIRO, Nicholas Engineers Research Corp.) for approximately 4 hours. The resulting product is a powder containing the chasteberry GPM complex.

### Example 3

### Preparation of Clover+Glycoprotein Matrix (GPM) Complex

This example demonstrates the preparation of clover plus glycoprotein matrix (GPM) complex to yield clover+GPM complex. The method employs preparing, in a first container, an aqueous solution of clover and adding a peptone made of amino acids.

In a second container an active yeast solution is prepared. Active baker's yeast, Saccharomyces cervisiae is added to water to form an aqueous solution. Maltose and gum acacia are then added.

The first container containing the clover is then inoculated very slowly into the active yeast solution to form a live fermented solution. The mixture is allowed to ferment for four to six hours. To promote yeast growth, plant proteins and carbohydrates are added. Proteolytic enzyme, such as papain, is then added.

Lactobacillus acidophillus is added to the live fermented solution and allowed to ferment for about 2 hours. Active fermentation is then stopped by heating the solution to 160-170° F. for three hours.

The fermented solution containing clover is then homogenized in a shearing pump (Charles Ross & Sons Corp.) for approximately 1-2 hours and spray dried (NIRO, Nicholas Engineers Research Corp.) for approximately 4 hours. The resulting product is a powder containing the clover GPM complex.

### Example 4

### Preparation of Black Cohosh+Glycoprotein Matrix (GPM) Complex

This example demonstrates the preparation of black cohosh plus glycoprotein matrix (GPM) complex to yield black cohosh+GPM complex. The method employs preparing, in a first container, an aqueous solution of black cohosh and adding a peptone made of amino acids.

In a second container an active yeast solution is prepared. Active baker's yeast, Saccharomyces cervisiae is added to water to form an aqueous solution. Maltose and gum acacia are then added.

The first container containing the black cohosh is then inoculated very slowly into the active yeast solution to form a live fermented solution. The mixture is allowed to ferment for four to six hours. To promote yeast growth, plant proteins and carbohydrates are added. Proteolytic enzyme, such as papain, is then added.

Lactobacillus acidophillus is added to the live fermented solution and allowed to ferment for about 2 hours. Active fermentation is then stopped by heating the solution to 160-170° F. for three hours.

The fermented solution containing black cohosh is then homogenized in a shearing pump (Charles Ross & Sons Corp.) for approximately 1-2 hours and spray dried (NIRO, Nicholas Engineers Research Corp.) for approximately 4 hours. The resulting product is a powder containing the black cohosh GPM complex.

What is claimed is:

1. A composition consisting essentially of a soy component, chasteberry, clover, and black cohosh, in a suitable carrier.

2. A composition according to claim 1, wherein the clover is red clover.

3. A composition according to claim 1, wherein the soy component is organic.

4. A composition according to claim 1, wherein the composition is in a form selected from the group consisting of tablets, gelatin capsules, pills, troches, elixirs, caplets, powders, granules, sachets, suspensions, syrups, wafers, and chewing gum.

5. A composition according to claim 1, wherein the composition is in a soft chew.

6. A composition consisting essentially of a soy component, chasteberry, clover, and black cohosh, in a suitable carrier, wherein each of the soy component, chasteberry, clover, and black cohosh is enrobed in a glycoprotein matrix.

7. A composition according to claim 6, wherein the clover is red clover.

8. A composition according to claim 6, wherein the soy component is organic.

9. A composition according to claim 6, wherein the composition is in a form selected from the group consisting of tablets, gelatin capsules, pills, troches, elixirs, caplets, powders, granules, sachets, suspensions, syrups, wafers, and chewing gum.

10. A composition according to claim 6, wherein the composition is a soft chew.

11. A composition according to claim 6, wherein the glycoprotein matrix is derived from one or more strains of microorganism.

12. A composition according to claim 11, wherein the microorganism is yeast.

13. A composition according to claim 12, wherein the yeast includes Saccharomyces cerevisiae.

14. A composition according to claim 11, wherein the microorganism is a probiotic.

15. A composition according to claim 14, wherein the probiotic includes Lactobacillus acidophilus.

16. A composition according to claim 14, wherein the probiotic includes Bacterium bifidus.

17. A method for promoting hormonal balance in a woman in need thereof comprising administering to the woman an effective amount of a composition consisting essentially of a soy component, chasteberry, clover, and black cohosh, in a suitable carrier.

18. A method according to claim 17, wherein the clover is red clover.

19. A method according to claim 17, wherein the soy component is organic.

20. A method according to claim 17, wherein the composition is administered orally.

21. A method for promoting hormonal balance in a woman in need thereof comprising administering to the woman an effective amount of a composition consisting essentially of a soy component, chasteberry, clover, and black cohosh, in a suitable carrier, wherein each of the soy component, chasteberry, clover, and black cohosh is enrobed in a glycoprotein matrix.

22. A method according to claim 21, wherein the clover is red clover.

23. A method according to claim 21, wherein the soy component is organic.
